(19) United States
(12) Patent Application Publication
(10) Pub. No.: US 2009/0027052 A1
(43) Pub. Date: Jan. 29, 2009

(54) Method for acquiring measured data

(76) Inventors: Jurgen Kampmeier, Erlangen (DE); Gunther Platsch, Rothenbach (DE); Martin Requardt, Nurnberg (DE); Stefan Roll, West Chester, PA (US); Sebastian Schmidt, Weisendorf (DE); Kristin Schmiedehausen, Buckenhof (DE); Michael Szimtenings, Bonn (DE)

Correspondence Address: HARNESS, DICKEY & PIERCE, P.L.C., P.O.BOX 8910, RESTON, VA, 20195, US

(21) Appl. No.: 12/213,233

(22) Filed: Jun. 17, 2008

(30) Foreign Application Priority Data
Jul. 4, 2007 (DE) 10 2007 030 962.9

(51) Int. Cl. G01R 33/483; A61B 5/055

(52) U.S. Cl. 324/309

(57) ABSTRACT

A PET examination which acquires a data record of the body of a patient is carried out during at least one embodiment of a method for acquiring measured data. On the basis of the measured values of the data record, at least one region of interest in the body of the patient is determined, in which at least one examination of at least one embodiment of a method is carried out.

### PRIORITY STATEMENT

The present application hereby claims priority under 35 U.S.C. §119 on German patent application number DE 10 2007 030 962.9 filed Jul. 4, 2007, the entire contents of which is hereby incorporated herein by reference.

### FIELD

Embodiments of the present invention generally relate to a method for acquiring measured data.

### BACKGROUND

The prevalence of positron emission tomography (PET), in addition to magnetic resonance imaging (MRI), has increased in medical diagnostics in recent years. Whereas MRI is an imaging method for displaying structures and slice images in the interior of the body, PET allows visualizing and quantifying metabolic activities in-vivo.

PET uses the particular characteristics of positron emitters and positron annihilation in order to quantitatively determine the function of organs or cell regions. In the process, appropriate chemical compounds (“radiopharmaceuticals”) marked with radionuclides are dispensed to the patient prior to the examination. During the decay process, the radionuclides emit positrons which interact with an electron after a short distance, leading to so-called annihilation. In this process, two gamma quanta are created, which fly apart in opposite directions (offset by 180°). The gamma quanta are detected by two mutually opposite PET detector modules within a determined time window (coincidence measurement), as a result of which the location of the annihilation is determined at a point on the connecting line between these two detector modules.

In general, the PET detector module must cover most of the gantry arch-length for detection purposes. The gantry arch-length is subdivided into detector elements having a side length of a few millimeters. Each detector element generates an event record on detection of a gamma quantum, indicating the time and the detection location, that is to say the corresponding detector element. This information is transmitted to fast logic and compared. If two events coincide within a maximum time interval, it is assumed that an annihilation process has taken place on the connecting line between the two associated detector elements. The reconstruction of the PET image is carried out using a tomography algorithm, that is to say so-called back projection.

In order to be able to answer diagnostic questions in detail, for example in cancer diagnosis, magnetic resonance (MR) spectroscopy is used to distinguish between normal and pathological tissue. This allows the composition of matter to be examined in a general form. The position of the resonance lines and their fine structure are influenced by the chemical surroundings of the excited nuclei. In general, this is referred to as chemical shift. As a result, different tissue types can be identified at different points in the human body by means of MR spectra. In particular, qualitative statements about the different types of tissue at different locations in the body are possible.

WO02/079801 A2 discloses a method which allows selection of a region of interest based on image reconstruction by way of SPECT, PET or CT. A biopsy is carried out in this area.

To acquire diagnostically relevant data of the body of a patient, different MR spectra are recorded from different examination volumes of the body of a patient. When selecting suitable measurement parameters, the fact that the examination volume excited to measure the spectrum must be as large as possible in order to obtain the highest signal-to-noise ratio possible in the spectra must first of all be taken into account. On the other hand, the examination volume should be as small as possible so that only one tissue type or only the focus of the ailment is imaged in the spectrum, without the surrounding healthy tissue. Satisfying both conditions ensures that the difference in the spectra between healthy and pathological tissue is as large as possible and these can thus be easily differentiated. Thus, precise differentiation between the different types of tissue is possible. However, the two conditions are contradictory, so that the optimum selection of the examination volume to be excited is often found to be difficult.

Different methods can be used for the examination. In the case of so-called single voxel spectroscopy, only single image elements of an MR image are selected for further examination; the examination volume is thus limited to one volume element. In the case of chemical shift imaging, spatial coding of the spectroscopic signal is carried out by means of a gradient system. This results in spectra distributed spatially in the form of a grid.

By way of example, MR images can be used to select the examination volumes. The anatomy of the patient visible in the MR images serves as a basis for selection, even if pathologies, i.e. tumors for example, can hardly be seen in the MR image. Therefore, there is little that can be used as a basis for optimum positioning and selection of volume elements. The size of the examination volumes is therefore often selected empirically.

The described difficulty of deliberate selection of examination volumes for subsequent examination does not only occur in the case of MR spectroscopy. The deliberate and best possible selection of the examination volume is also important in the case of so-called functional MR imaging (fMRI), diffusion maps, T1- and T2-weighted images and quantitative parameter maps. Further examination methods, such as computed tomography (perfusion measurement, multi-energy imaging) or x-rays may likewise benefit from targeted volume selection.

### SUMMARY

In at least one embodiment of the invention, an improved method is provided by which measured data can be acquired from deliberately selected examination volumes of the human body.

In at least one embodiment, a PET examination is carried out first of all in order to acquire a data record of the body of the patient. At least one region of interest in the body of the patient is determined on the basis of the acquired PET data and the measured values of the data record. Subsequently, an examination method is applied to the at least one region of interest. Alternatively, measured data which is already available from an examination method within the region of interest can also be evaluated. In both cases, the use of PET data to determine the region of interest has the advantage that various pathologies can be imaged in a highly specific manner by means of PET imaging. This comprises both structural and chemical abnormalities, the boundaries of which can be displayed.

In particular, using highly specific PET biomarker, such as e.g. marked amino acids, pathological sections can, for example, be delimited well from the surrounding tissue of the brain of a patient. In this manner, regions of interest having pathologies can be selected highly specifically and further examinations can then be carried out on them. The uncertainty existing for positioning on the basis of conventional magnetic resonance images because of the poor capability to differentiate between different regions is not present or is at least greatly reduced in the case of PET images.

In one advantageous embodiment of the invention, the examination carried out using MR spectroscopy. Using this, pathological tissue types can be distinguished in more detail.

Further advantageous embodiments of the invention relate in particular to the definitions of the region of interest. This can be carried out both interactively by a user on the PET data record, and by means of automatic segmentation. In this case, the PET data record is analyzed, and divided into various regions of differing signal intensity in an automated fashion. Since the signal intensity of pathological tissue is particularly high or low in the PET data record, cohesive regions are obtained in this manner for the subsequent examination. In an alternative method, the region is determined by finding cohesive measured values which differ significantly from a reference value. The subsequent examination is then carried out in regions with cohesive measured values of different intensity.

A region defined in this way can be formed in both two and three dimensions.

In a further alternative, the identification of the regions of interest from the PET data record can be carried out by comparing the PET data record with reference data records. By way of example, these can be contained in an atlas of PET records acquired from healthy persons. This makes it possible to quickly identify whether pathological tissue is present here, in contrast to the healthy tissue stored in the atlas of the PET data records.

In a further advantageous refinement of the invention, the information from the PET record is used in order to suggest further parameters for subsequent examination to the user. By way of example, in addition to the position suggested in any case, particular magnetic resonance sequences can be suggested which are especially tailored to the selected body region or the ailment to the examined.

### BRIEF DESCRIPTION OF THE DRAWINGS

Further advantages and refinements of the invention result from the example embodiments described below with reference to the figures, in which

FIG. 1 shows a schematic representation of a combined MR-PET appliance, and

FIG. 2 shows a schematic flowchart of one preferred example embodiment of the method.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

The example embodiments of the invention are preferably used in conjunction with a combined MR-PET appliance. A combined appliance has the advantage that both MR and PET data can be acquired isocentrically. This allows precise definition of the examination volume within the region of interest from the data of the first modality (PET) and the use of this information in the further modality (e.g. magnetic resonance). Although transfer of the volume information of the region of interest from an external PET appliance to an MR appliance is possible, this involves increased complexity to register the data. In most cases, the registration is very imprecise due to the lack of representation of anatomical structures in the PET data as a function of the selected radiopharmaceutical and body region.

The example embodiments of the invention described in detail are not limited to the measurement of spectroscopy data. In general, all data which is determinable by magnetic resonance or other imaging methods can be determined in the region of interest selected from the PET data records. By way of example, fMRI data, diffusion maps, T1- or T2-weighted images or quantitative parameter maps can be acquired from the region of interest by means of magnetic resonance examinations, instead of using spectroscopy data. Methods from computed tomography (e.g. perfusion measurement, multi-energy imaging) or x-ray imaging can likewise be used.

In each case, one advantage of the described method is that the PET data record can be used very specifically to restrict the region of interest to a particular pathology that is present in the patient.

Additionally, it is however also possible to display different biological characteristics in the PET data record by using a plurality of so-called tracers, and thus further to optimize the region of interest and the volume defined in this way by this, or select a plurality of different examination volumes in one go, which are then analyzed in the subsequent examinations.

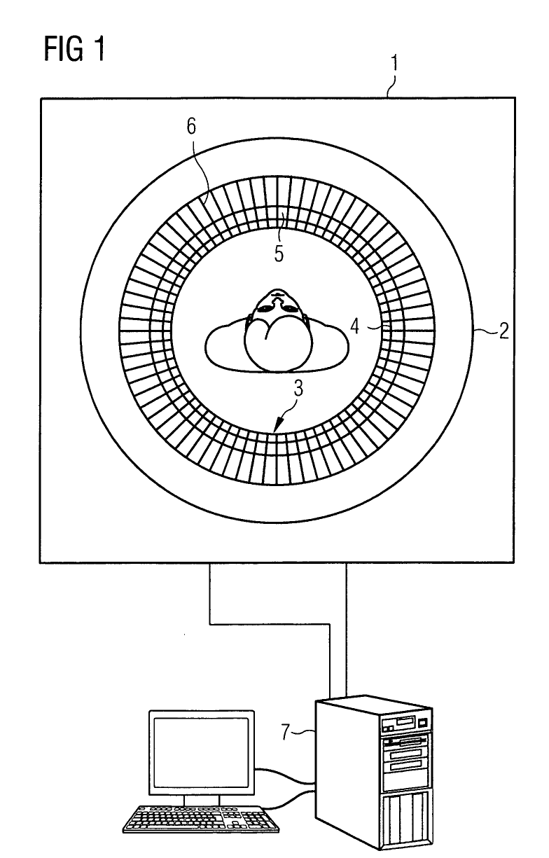

FIG. 1 shows a known device 1 for superposed MRI and PET imaging. The device 1 comprises a known MRI tube 2.

The MRI tube 2 defines a longitudinal direction z, which extends orthogonally with respect to the plane of the drawing of FIG. 1.

As shown in FIG. 1, a plurality of PET detector units 3, which are arranged in pairs opposite each other about the longitudinal direction z, are arranged coaxially within the MRI tube 2. The PET detector units 3 preferably comprise an APD photodiode array 5 having an upstream array of LSO crystals 4 and an electrical amplifier circuit (AMP) 6. However, embodiments of the invention are not limited to PET detector units 3 having the APD photodiode array 5 and the upstream array of LSO crystals; in fact, different types of photodiodes, crystals and apparatuses can be used equally well for the purpose of detection.

The image processing for superposed MRI and PET imaging is carried out by a computer 7.

The MRI tube 2 defines a cylindrical first field of view along its longitudinal direction z. The multiplicity of PET detector units 3 defines a cylindrical second field of view along the longitudinal direction z. According to an embodiment of the invention, the second field of view of the PET detector units 3 substantially corresponds to the first field of view of the MRI tube 2. This is implemented by a corresponding adaptation of the arrangement density of the PET detector units 3 along the longitudinal direction z.

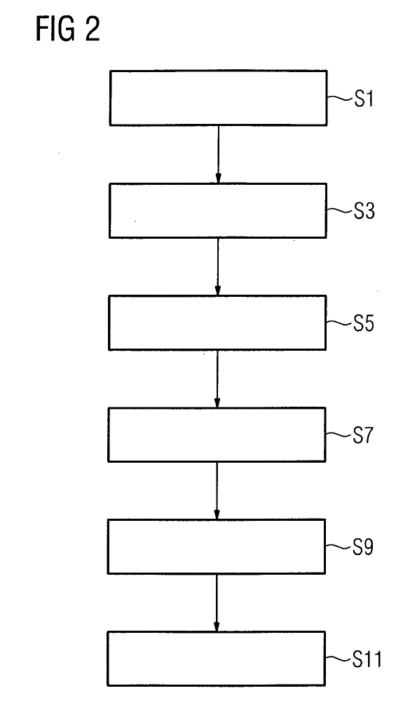

FIG. 2 shows a schematic flowchart of one example embodiment of the invention. In this case, a radiopharmaceutical and a PET biomarker particularly suited to the present specific pathology to be imaged, for example a tumor, are dispensed to the patient in a first method step S1. In a second method step S3, the patient is placed in a combined PET-MR appliance and a PET data record is recorded. The PET data record is evaluated in a third method step S5. In the process, one or more regions of interest having a high signal intensity and corresponding examination volumes are defined by way of the methods described previously, that is to say either by manual segmenting performed by the user or by comparison with reference values. In a fourth method step S7, a magnetic resonance examination method is selected by the user. Alternatively, a magnetic resonance examination method can be suggested by the system. By way of example, this suggestion can be based on a suspected or already diagnosed ailment of the patient which will be diagnosed in more detail by means of the examination. In a fifth method step S9, the selected examination method is set. In the process, measurement parameters are automatically suggested by the system.

By way of example, these measurement parameters are stored in a database and are assigned there to specific body regions or conditions. Depending on the position of the region of interest in the PET data record and, optionally, the suspected ailment defined by the user, appropriate parameters for the magnetic resonance examination are selected and presented to the user on a computer monitor, for example. The actual magnetic resonance examination of the regions of interest is carried out in a sixth method step S11. Measured data is acquired in the process which provides information about the pathology in the respective region of interest. By way of example, this allows specific diagnosis of the course of an ailment.

Alternatively, it is possible to carry out the MR examination and the PET examination simultaneously. In this case, it is not the MR data recording but the evaluation of the MR data which is influenced by the results of the PET examination.

There are a number of possibilities for carrying out the magnetic resonance examination. For example, single voxel spectroscopy can be carried out in order to examine the pathology of the tissue located in the region of interest in more detail. In this case, the volume to be examined can be defined to be a cube which surrounds the volume of the region of interest defined with aid of the PET data record as precisely as possible. Alternatively, a volume of arbitrary shape can be excited in the case of single volume spectroscopy, by way of suitable radio-frequency pulses. These particular pulses are emitted at the same time as the pulses from the gradient system. Similar to the spatial coding for reception of an RF signal, this can result in the RF signal radiating inward exciting only a previously determined volume. In this context, it is particularly advantageous for measurements to be carried out using a parallel transmitter chain, since the duration of the excitation pulses can be decreased in this manner (Transmit-SENSE). The method is based on excitation being carried out simultaneously by more than one radio-frequency coil.

In the case of chemical shift imaging, both the position of the grid of the volume elements and the size of the volume elements can be adapted prior to the recording of data, in such a way that the volume elements are either completely contained by the volume defined with aid of the PET data record or are located completely outside said volume. In these cases, the volume elements are still cubic.

In the case of chemical shift imaging, it is likewise possible to optimize the excitation sequence in such a way that the shape of the volume elements corresponds to the volume defined in the PET data record. In this case, the volume need no longer be cubic. SLOOP (spatial localization with optimal pointspread function) is one possible technique for achieving this. The position of the grid of the volume elements can be chosen arbitrarily during data reconstruction using chemical shift imaging. This can be used in particular to match the edges of the volume elements to the edges of the volume defined in the PET data record. This allows the greatest possible spatial accuracy of the magnetic resonance examinations. It is likewise possible to define a further region after the reconstruction and to average the volume elements in this region, resulting in a sum spectrum. The region in which the volume elements are to be averaged can likewise be defined with the aid of the PET data record.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for acquiring measured data, comprising:
carrying out a PET examination and acquiring a data record of the body of a patient;
automatically determining at least one region of interest in the body of the patient based upon measured values in the acquired data record; and
carrying out at least one imaging examination method to acquire the measured data within the at least one region of interest.

2. A method for evaluating measured data, comprising:
carrying out a PET examination and acquiring a data record of the body of a patient;
determining at least one region of interest in the body of the patient based upon measured values in the acquired data record; and
evaluating the measured data from an examination method within the at least one region of interest.

3. The method as claimed in claim 1, wherein the examination method is a magnetic resonance examination.

4. The method as claimed in claim 3, wherein spectroscopy is carried out during the course of the magnetic resonance examination.

5. The method as claimed in claim 1, wherein the determination of the region of interest is carried out by locating cohesive measured values which differ from a reference value.

6. The method as claimed in claim 5, wherein the shape of the region of interest is matched to the shape of the cohesive measured values.

7. The method as claimed in claim 1, wherein the determination of the region of interest is carried out by an automatic segmentation.

8. The method as claimed in claim 1, wherein the determination of the region of interest is carried out by comparison of the data record with a reference data record and locating differences from the latter.

9. The method as claimed in claim 1, wherein measurement parameters are defined for the examination method based upon the measured values located in the region of interest.

10. The method as claimed in claim 1, wherein the measured data within the region of interest are averaged.

11. The method as claimed in claim 2, wherein the examination method is a magnetic resonance examination.

12. The method as claimed in claim 11, wherein spectroscopy is carried out during the course of the magnetic resonance examination.

13. The method as claimed in claim 2, wherein the determination of the region of interest is carried out by locating cohesive measured values which differ from a reference value.

14. The method as claimed in claim 13, wherein the shape of the region of interest is matched to the shape of the cohesive measured values.

15. The method as claimed in claim 2, wherein the determination of the region of interest is carried out by an automatic segmentation.

16. The method as claimed in claim 2, wherein the determination of the region of interest is carried out by comparison of the data record with a reference data record and locating differences from the latter.

17. The method as claimed in claim 2, wherein measurement parameters are defined for the examination method based upon the measured values located in the region of interest.

18. The method as claimed in claim 2, wherein the measured data within the region of interest are averaged.

19. A computer readable medium including program segments for, when executed on a computer device, causing the computer device to implement the method of claim 1.

20. A computer readable medium including program segments for, when executed on a computer device, causing the computer device to implement the method of claim 2.
